(19) United States
(12) Patent Application Publication
(10) Pub. No.: US 2008/0160083 A1
(43) Pub. Date: Jul. 3, 2008

(54) COMPOSITION AND METHOD FOR TREATMENT OF PREMENSTRUAL SYMPTOMS

(76) Inventor: Jarret Morrow, Boca Raton, FL (US)

Correspondence Address: ROBERT M. SCHWARTZ, P.A., P.O. BOX 221470, HOLLYWOOD, FL, 33022, US

(21) Appl. No.: 11/968,246

(22) Filed: Jan. 2, 2008

Related U.S. Application Data
(60) Provisional application No. 60/883,204, filed on Jan. 3, 2007.

(51) Int. Cl. A61K 9/28; A61K 33/06; A61P 15/00; A61K 33/08

(52) U.S. Cl. 424/474; 424/682; 424/692

(57) ABSTRACT

The present invention is a composition delivering effective amounts of Chasteberry Extract, Pyridoxine, and Magnesium in a single dosage unit and a method of treating PMS and related disorders with the composition.

### INDEX TO RELATED APPLICATIONS

This application claims the benefit of U.S. Patent Application Ser. No. 60/883,204, filed Jan. 3, 2007, the disclosure of which is incorporated by reference in its entirety.

### BRIEF SUMMARY OF THE INVENTION

The present invention relates to a novel combination of Chasteberry extract, Pyridoxine (Vitamin B-6), and Magnesium. The dosage form provides greater efficacy than previous combinations and products.

Vitex agnus castus is also known as Chasteberry Tree and Monks Pepper. It is typically found in Mediterranean Countries. The Pharmacology is not exactly known but early investigations have theorized that Vitex contained progesterone—like compounds.

The herb acts at the pituitary gland.

Administration of Chasteberry results in the release of follicle stimulating hormone (FSH) being blocked while the secretion of luteinising hormone (LH) and prolactin is stimulated. A net effect is a shift in the balance in the production of estrogen to progesterone.

Evidence from clinical trials (initially not randomized double blind trials) supports a progesterogenic effect. (Herbal Medicines 1994 Anderson and Phillipson) In vitro work on the extract has demonstrated inhibition of prolactin secretion from rat pituitary cell cultures (Zeitschrift fur Phytotherapie 12, 77-82, 1991).

Chemical analysis has demonstrated the presence of Flavonoids: flavanol (Kaempferol, quercetagetin) derivatives, casticin, isovitexin, isoorientin, luteolin 7 glucoside and the Iridoids Aucubin and agnuside.

The flavonoid Casticin, and the Iridoids Aucubin and Agnuside are believed to be the major contributors to the pharmacological effects of the extract. The formula for each is set forth below:

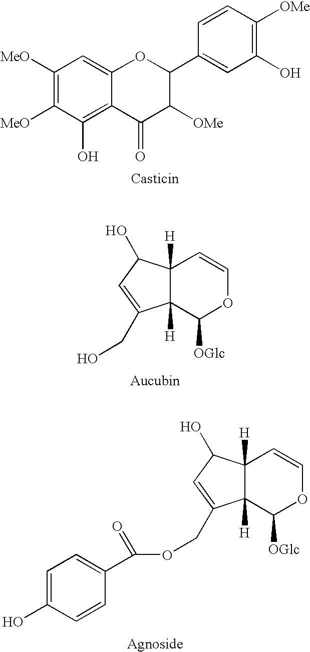

Some Iridoids are known to be stimulants, anxiolytics and anti-inflammatory.

Casticin, Aucubin and Agnuside have anti-inflammatory activity, sedative and hepatoprotective properties.

There is also no accepted standard for quality control of Chasteberry extract.

The quality of the herbal material used in nutraceutical products needs to take into account the following aspects of the plant material: Macroscopic and microscopic properties, BOTANICAL name and family, and geographical source, the method of drying and/or extraction (including solvents used etc.), and consistent analytical data (TLC, HPLC etc.).

There are currently various extracts that purport to standardize the extract with varying criteria.

Pyridoxine also known has Vitamin B-6 and the chemical names 4,5-bis(hydroxymethyl)-2-methylpyridin-3-ol; or 5-hydroxy-6-methyl-3,4-pyridinedimethanol; has a chemical formula of C₈H₁₁NO₃ a molecular weight of 169.18 and a structure as set forth below:

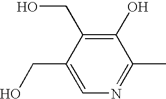

Pyridoxine is often used as the hydrochloride salt. Vitamin B₆ has become a popular remedy for treating the discomforts associated with premenstrual syndrome (PMS). (Johnson SR. Premenstrual syndrome therapy. Clin Obstet Gynecol 1998; 41:405-21).

A concern with vitamin B₆ therapy is toxicity has been seen in increasing numbers of women taking vitamin B₆ supplements for PMS. One review indicated that neuropathy was present in 23 of 58 women taking daily vitamin B₆ supplements for PMS whose blood levels of B₆ were above normal (Dalton K. Pyridoxine overdose in premenstrual syndrome. Lancet 1985; 1, May 18:1168.)

The Food and Nutrition Board of the Institute of Medicine has established an upper tolerable intake level (UL) for vitamin B₆ of 100 mg per day for all adults (Institute of Medicine-Food and Nutrition Board. Dietary Reference Intakes: Thiamin, riboflavin, niacin, vitamin B6, folate, vitamin B12, pantothenic acid, biotin, and choline. National Academy Press. Washington, DC, 1998.).

Magnesium is a necessary nutriant for humans. Green vegetables such as spinach provide magnesium because the center of the chlorophyll molecule contains magnesium. Nuts (especially almonds), seeds, and some whole grains are also good sources of magnesium. Although magnesium is present in many foods, it usually occurs in dilute form. As with most nutrients, daily needs for magnesium are unlikely to be met from a single serving of any single food. Eating a wide variety of foods, including five servings of fruits and vegetables daily and plenty of whole grains, helps to ensure an adequate intake of magnesium.

Because magnesium readily dissolves in the water used to refine foods, and also in the water-rich parts of certain foods which are removed during refining, the magnesium content of many refined foods is low. Whole-wheat bread, for example, has twice as much magnesium as white bread because the magnesium-rich germ and bran are removed when white flour is processed. The table of food sources of magnesium suggests many dietary sources of magnesium.

Water can provide magnesium, but the amount varies according to the water supply. “Hard” water contains more magnesium than “soft” water. Dietary surveys do not estimate magnesium intake from water, which may lead to underestimating total magnesium intake and its variability.

Too much magnesium may make it difficult for the body to absorb calcium. Not enough magnesium can lead to hypomagnesemia as described above, with irregular heartbeats, high blood pressure (a sign in humans but not some experimental animals such as rodents), insomnia and muscle spasms (fasciculation). However, as noted, symptoms of low magnesium from pure dietary deficiency are thought to be rarely encountered. The U.S. reccommended daily amount is 400 mg of magnesium per day.

It has been well established that Magnesium contributes to increased hepatic metabolism. (Peters and Fouts A STUDY OF SOME POSSIBLE MECHANISMS BY WHICH MAGNESIUM ACTIVATES HEPATIC MICROSOMAL DRUG METABOLISM IN VITRO Journal of Pharmacology And Experimental Therapeutics, Vol. 173, Issue 2, 233-241, 1970).

One typical source of magnesium as a supplement is magnesium oxide (MgO).

There is a need to provide a nutritional approach to ameliorate the physical and psychological symptoms of PMS and relatedd disorders.

The present invention addresses this need by providing a single unit dosage form wherein Magnesium, Pyridoxine and Chasteberry extract in certain ratios and dosage levels provide the desired therapy.

This is advantageous because it becomes less expensive to provide the desired therapy and a single dosage form increases patient compliance with the therapy regimen.

In addition to being suitable for PMS therapy, the single unit dosage form of the present invention is also contemplated to provide therapy for Premenstrual depression, Premenstrual dysphoric disorder (PMDD), Cyclical breast discomfort or mastodynia, minimize fibrocystic breast symptoms, control menstrual related acne, relieve pain of endometriosis, and to treat general menopausal difficulties.

In one embodiment the ratio of Chasteberry:Pyridoxine:Magnesium is between 1:2.5-4:15-20. In a preferred embodiment, the ratio is 1:2.5-4:15-20. Still another preferred embodiment is about 1:3.3:17. The dual mechanism of magnesium to increase hepatic metabolism along with an elevated dose of Pyridoxine actuates the threrapeutic properties of both the standardized extract in general and specifically Casticin. When the ACTIVES are in combination in specific ratios, they provide for a single dosage form that allows increased effacacious threapy to ocurr. There are many dosage forms known in the art, as detailed below. A preferred dosage form is a tablet.

In one embodiment the present invention comprises
An oral dosage form comprising;
   (a) Chasteberry Extract standardized to 0.6% of Casticin;
   (b) Pyridoxine;
   (c) Magnesium; in a ratio of (a):(b):(C) of 1:2-5:12-25.

In a preferred embodiment the dosage form is a single unit dosage form. In a preferred embodiment, Chasteberry Extract standardized to 0.6% of Casticin.

In a preferred embodiment Pyridoxine is present in a salt form that may be any sulfate, phosphate, carbonate, oxalate, chloride, fluoride, bromide, or other acceptable salts. A preferred salt is the hydrochloride.

In a preferred embodiment Magnesium is present in a salt form that may be any sulfate, phosphate, carbonate, oxalate, chloride, fluoride, bromide, oxide, or other acceptable salts. A preferred salt is the oxide.

The dosage form may be any dosage form acceptable for delivery of a therapeutic substance to a patient. The compositions can be provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a buccal or sublingual solid, a granule, a film, a sprinkle, a pellet, a bead, a pill, a powder, a triturate, a platelet, a strip or a sachet. Compositions can also be administered as a “dry syrup”, where the finished dosage form is placed directly on the tongue and swallowed or followed with a drink or beverage. These forms are well known in the art and are packaged appropriately. The compositions can be formulated for oral, nasal, buccal, or transmucosal, delivery, although oral delivery is presently preferred.

Most preferred is a dosage form that is a tablet or capsule.

In a preferred embodiment, the dosage form further comprises an enteric coating.

It is also preferred that the dosage form be provided as a single dosage unit.

It is an object of the present invention of the present invention to provide therapeutic levels of Chasteberry, Pyridoxine, and Magnesium in a single dosage form.

### DETAILED DESCRIPTION OF THE PREFERRED EMBODIMENTS

The present invention provides for a single unit dosage form delivering improved therapy of a combination of Chasteberry Extract, Pyridoxine, and Magnesium.

The present invention has found an improved combination of therapy when using Chasteberry extract standardized to 0.6% Casticin. As used herein, standardized extract will refer to will refer to Chasteberry extract standardized to 0.6% Casticin. As used herein, ACTIVES refers to the combination of Chasteberry Extract-standardized to 0.6% Casticin, Pyridoxine, and Magnesium. Previous dosage forms have used Chasteberry extract in amounts up to 200mg per dose. The present invention has discovered the standardized extract provides enhanced therapeutic effects when administered in specific ratio combination with an elevated dose of Pyridoxine and a standard dose of Magnesium. Thus a dose of the present invention may be 50 mg or less. In a preferred embodiment, the standardized extract is present in 20mg or less per dose.

The elevated dose of Pyridoxine is less than 100 mg upper tolerable limit as set forth by the Institute of Medicine-Food and Nutrition Board. In a preferred embodiment, the elevated dose of Pyridoxine is less than 75 mg. In another preferred embodiment, the elevated dose of Pyridoxine is less than 70 mg. A preferred form Pyridoxine is the hydrochloride salt.

Current guidelines in the US provide a recommended daily amount of Magnesium is 400 mg. In a preferred embodiment, Magnesium is included in the unit dosage in an amount less than 400 mg. It has been well established that Magnesium contributes to increased hepatic metabolism.

The present formulation has found that the increased hepatic effect of magnesium provides greater sympathetic effect of the Casticin and Pyridoxine and allows for higher adsorption than previous formulations. A difficulty in arriving at the formulation of the present invention was determining a therapeutically ratio of the ACTIVES and delivering the ACTIVES such that they individually do not exceed the established safe and tolerable limits. During formulation development, it was determined that an elevated amount of Pyridoxine would be required to produce the desired results. The elevated amount of Pyridoxine required in the formulation of the present invention is acceptable because the amount is sufficient to contribute to the desired function of the combined ACTIVES and below the established limits.

The compositions of the present invention can be processed by agglomeration, air suspension chilling, air suspension drying, balling, coacervation, coating, comminution, compression, cryopelletization, encapsulation, extrusion, wet granulation, dry granulation, homogenization, inclusion complexation, lyophilization, melting, microencapsulation, mixing, molding, pan coating, solvent dehydration, sonication, spheronization, spray chilling, spray congealing, spray drying, or other processes known in the art.

The composition can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition.

The term “enteric coating” as used herein relates to a mixture of pharmaceutically acceptable excipients that is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Alternatively, an enteric coating may be applied in an aqueous/organic cosolvent system. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Cellulose Derivatives are a preferred enteric coat material. Examples of suitable cellulose derivatives are: ethyl cellulose; reaction mixtures of partial acetate esters of cellulose with phthalic anhydride.

A preferred coating is aqueous Ethylcellulose Dispersion. The dispersion is a combination of film-forming polymer; plasticizer and stabilizers. Designed for sustained release and taste masking applications, the dispersion provides the flexibility to adjust drug release rates with reproducible profiles that are relatively insensitive to pH.

The principal means of drug release is by diffusion through the dispersion membrane and is directly controlled by film thickness. Increasing or decreasing the quantity of dispersion applied can easily modify the rate of release.

Two well-known dispersions are Surelease (Colorcon, West Point, Pa.) and Aquacoat ECD (FMC).

The performance of a coating can vary based on the degree and type of substitution. Cellulose acetate phthalate (CAP) dissolves in pH>6. Aquateric (FMC) is an aqueous based system and is a spray dried CAP psuedolatex. Other components in Aquateric can include pluronics, Tweens, and acetylated monoglycerides; cellulose acetate trimellitate (Eastman); methylcellulose (Pharmacoat, Methocel); hydroxypropyl methyl cellulose phthalate (HPMCP). The performance can vary based on the degree and type of substitution. HP-50, HP-55, HP-55S, HP-55F grades are suitable; hydroxypropyl methyl cellulose succinate (HPMCS; AQOAT (Shin Etsu)).

The coating can, and usually does, contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

A coating process frequently involves spraying a coating solution onto a substrate. The coating solution can be a molten solution of the encapsulation coat composition free of a dispersing, medium. The coating solution can also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level utilizing appropriate drying processes, such as vacuum evaporation, heating, freeze drying, etc.

Solvent-based coating is when the components of the invention are solubilized and/or dispersed in a solvent. The solvent can be aqueous. When the solvent is aqueous-based, the components can be emulsified with an appropriate emulsifier, organic solvent, or a supercritical fluid. Solvents with a lower melting point than water and higher evaporation numbers are preferred. Solvent mixtures with other organic solvents or water are often employed to get appropriate viscosity and component solubilization. Typical solvents include ethanol, methanol, isopropanol, acetone, dichloromethane, trichloromethane and ethyl acetate. Appropriate polymers can also be added as needed. Cellulosic derivatives and polymethacrylates are particularly suitable additives for organic solvent coating. Dissolution and solubilization of the components is facilitated by rigorous stirring or heating. Plasticizers may be also be added to stimulate dissolution. Colorants and antisticking agents can be employed as needed.

The following are presented by way of example and are not intended to limit the scope of the invention.

One general formulation is as follows:

| | |
|---|---|
| Chasteberry Extract | 1-10% |
| Pyridoxine | 5-25% |
| Magnesium | 30-70% |
| Filler | 20-75% |
| Binder | 1-20% |
| Disintegrant | up to 15% |
| Lubricant | up to 10% |
| Glident | up to 10% |

### EXAMPLE 1

In one embodiment a first blend comprising 25g Chasteberry Extract, 75 g Pyridoxine, and 400 g of Magnesium Oxide are passed through a 25 mesh screen and blended until uniformly mixed. A second blend is prepared comprising 250 g microcrystalline cellulose (a common form sold as AVICEL® by FMC, Philadelphia, Pa.), 10 g stearic acid, and 10 g croscarmellose sodium are each passed through a 25 mesh screen. The first and second blends are combined in a v-blender and mixed 45 minutes or long enough to ensure content uniformity as is commonly known and practiced in the art. The blender is stopped and 5 mg of silicon dioxide and 10 mg of magnesium stearate are screened through a 25 mesh screen and added to the blender. The mixture is blended an additional five minutes. The tableting mixture is discharged from the blender. Capsule shaped tablets with a target weight of 700 mg (±6%) are compressed with a target hardness of 8-12 kP.

Tablets prepared according to Example 1 may optionally be coated with a layer. Alternatively, the tablets may be coated with more than one layer. Any layer may be functional or non-functional and may include, but would not be limited to controlled release, delayed release, sustained release, color, taste masking, moisture barrier, or any other layer disposed on the surface as are commonly practices in the art. In a preferred embodiment, the tablets are coated with an enteric layer such that they do not dissolve in the gastric pH of approximately 1.2.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An oral dosage form comprising;
(a) Chasteberry Extract standardized to 0.6% of Casticin;
(b) Pyridoxine;
(c) Magnesium;
in a ratio of (a):(b):(C) of 1:2-5:12-25.

2. The dosage form of claim 1 having a ratio of 1:2.5-4:15-20.

3. The dosage form of claim 1 wherein Pyridoxine is present as a salt, isomer, or derivative.

4. The dosage form of claim 1 wherein Pyridoxine is present as the hydrochloride.

5. The dosage form of claim 1 wherein Magnesium is present in a salt, isomer, or derivative.

6. The dosage form of claim 1 wherein Magnesium is present as a sulfate, phosphate, carbonate, or oxide.

7. The dosage form of claim 1 wherein Magnesium is present as the Magnesium Oxide salt.

8. The dosage form of claim 1 wherein said dosage form is a tablet or capsule.

9. The dosage form of claim 1 wherein said dosage form further comprises an enteric coating.

10. The dosage form of claim 1 wherein said dosage form is provided as a single unit.

11. The dosage form of claim 1 comprising:
(a) Chasteberry Extract 1-15%;
(b) Pyridoxine 5-25%;
(c) Magnesium 30-70%;
based on the total weight of the dosage form.

12. The dosage form of claim 1 comprising a coating disposed on said dosage form.

13. The dosage form of claim 13 wherein said coating is at least one layer.

14. The dosage form of claim 13 wherein said layer is functional or non-functional.

15. The dosage form of claim 1 comprising a functional layer selected from controlled release, sustained release, delayed release, taste masking, or moisture control.

16. The dosage form of claim 1 comprising an enteric layer.

17. A method of providing therapy to a patient comprising the steps of:
(a) preparing a single unit dosage form comprising
(i) Chasteberry Extract 1-15%;
(ii) Pyridoxine 5-25%;
(iii) Magnesium 30-70%;
said Chasteberry Extract, Pyridoxine, and Magnesium being present in a ratio of 1:2-5:12-25 and being present in any form including salts, isomers, and derivatives;
(b) administering said single unit dosage form to a patient.

18. A method for providing therapy to treat, prevent or ameliorate PMS, Premenstrual depression, Premenstrual dysphoric disorder (PMDD), Cyclical breast discomfort, fibrocystic breast symptoms, menstrual related acne, endometriosis, and treat general menopausal difficulties, comprising the steps of:
(a) preparing a single unit dosage form comprising
(i) Chasteberry Extract 1-15%;
(ii) Pyridoxine 5-25%;
(iii) Magnesium 30-70%;
said Chasteberry Extract, Pyridoxine, and Magnesium being present in a ratio of 1:2-5:12-25 and being present in any form including salts, isomers, and derivatives;
(b) administering said single unit dosage form to a patient.
